(19) United States
(12) Patent Application Publication
(10) Pub. No.: US 2020/0305837 A1
(43) Pub. Date: Oct. 1, 2020

(54) SYSTEM AND METHOD FOR GUIDED ULTRASOUND IMAGING

(71) Applicant: GENERAL ELECTRIC COMPANY, Schenectady, NY (US)

(72) Inventors: Chandan Kumar Mallappa Aladahalli, Bangalore (IN); Krishna Seetharam Shriram, Bangalore (IN); Srinivas Varna, Bangalore (IN)

(21) Appl. No.: 16/366,371

(22) Filed: Mar. 27, 2019

(51) Int. Cl. A61B 8/00; A61B 8/08

(52) U.S. Cl. CPC A61B 8/4461; A61B 8/463; A61B 8/08; A61B 8/5207

(57) ABSTRACT

An ultrasound scanning guidance method includes acquiring an image by an ultrasound probe of a target organ during an ultrasound scanning procedure. The acquired image corresponds to a pose of the target organ in an acquired scan plane. The method further includes processing the image by a guidance unit to determine an anatomical context around the target organ based on the acquired image. The processing the image also includes determining a relative location of the acquired scan plane with reference to a standard scan plane based on the pose of the target organ and the anatomical context. The processing further includes generating scanning guidance based on the relative location of the acquired scan plane. The scanning guidance includes information to move the probe towards a standard pose of the target organ. The method also includes presenting the scanning guidance by an output device for aiding continuance of the scanning procedure.

### BACKGROUND

Embodiments of the present specification relate generally to ultrasound imaging, and more particularly to systems and methods for guided ultrasound imaging without the aid of sensors.

Ultrasound imaging is a medical imaging technique that may be used to view three-dimensional structures inside the body of a living being. Ultrasound images, captured in real-time, also enable visualization of movement of the internal organs, blood flowing through the blood vessels and the stiffness of tissue. Ultrasound imaging employs high-frequency sound waves. Since these high-frequency sound waves are not ionizing radiation, prolonged usage of ultrasound imaging does not cause internal organ/tissue damage. During an ultrasound exam, a transducer, commonly referred to as a probe, is placed directly on the skin of a subject for acquiring ultrasound imagery. A thin layer of gel is applied to the skin so that the ultrasound waves are transmitted from the transducer through the medium of the gel into the body. The ultrasound image is produced based upon a measurement of the reflection of the ultrasound waves off the body structures. The strength of the ultrasound signal, measured as the amplitude of the detected sound wave reflection, and the time taken for the sound waves to travel through the body provide the information necessary to compute an image.

Ultrasound imaging provides several advantages that include providing real-time images. The ultrasound imaging equipment is portable and lower in cost. However, the quality of the ultrasound images depends mainly on the skill of the operator performing the ultrasound scanning. A good quality ultrasound image depends among other things, on factors such as placement of the probe on the body, spatial orientation of the probe, and choice of movements of the probe on the organ. Generally, the ultrasound operator is provided with the visual feedback of the imagery produced during the ultrasound. The operator needs to interpret the image and based on his/her experience, decide to change the placement and orientation of the probe and determine its further movement for acquisition of relevant ultrasound data. Thus, essentially, the navigation of the probe is a manual process consisting of an iterative trial and error approach guided by operator's skills. Consequently, enabling a relatively less experienced user to acquire clinically correct ultrasound images requires guidance due to large variability in patient anatomy, size and tissue characteristics. Movement of the patient during scanning may compound the problem. State of the art solutions entail use of expensive sensors to estimate the location of the probe to guide the user. However, it may not be desirable to use expensive sensors in low cost devices. Therefore, alternate techniques are desired to provide guidance to a less experienced user.

### BRIEF DESCRIPTION

In accordance with one aspect of the present specification, an ultrasound scanning guidance method is disclosed. The method includes acquiring an image by an ultrasound probe of a target organ during an ultrasound scanning procedure. The acquired image corresponds to a pose of the target organ in an acquired scan plane. The method further includes processing the image by a guidance unit to determine an anatomical context around the target organ based on the acquired image. The processing the image by the guidance unit includes determining a relative location of the acquired scan plane with reference to a standard scan plane based on the pose of the target organ and the anatomical context. The processing further includes generating scanning guidance based on the relative location of the acquired scan plane. The scanning guidance includes information to move the probe towards a standard pose of the target organ. The method also includes presenting the scanning guidance by an output device for aiding continuance of the scanning procedure.

In accordance with another aspect of the present specification, an ultrasound scanning system is disclosed. The system includes an ultrasound probe configured to acquire an image of a target organ during an ultrasound scanning procedure. The acquired image corresponds to a pose of the target organ in an acquired scan plane. The system further includes a guidance unit communicatively coupled to the ultrasound probe and configured to determine an anatomical context around the target organ based on the acquired image. The guidance unit is further configured to determine a relative location of the acquired scan plane with reference to a standard scan plane based on the pose of the target organ and the anatomical context. The guidance unit is also configured to generate a scanning guidance based on the relative location of the acquired scan plane. The scanning guidance includes information to move the probe towards a standard pose of the target organ. The system further includes an output device communicatively coupled to the guidance unit and configured to present the scanning guidance for aiding continuance of the scanning procedure.

### DRAWINGS

These and other features and aspects of embodiments of the present invention will become better understood when the following detailed description is read with reference to the accompanying drawings in which like characters represent like parts throughout the drawings, wherein:

FIG. 1 is a diagrammatic illustration of a system for guided ultrasound imaging in accordance with an exemplary embodiment;

FIGS. 2A and 2B illustrate images of a current scan plane in comparison with a target scan plane in accordance with an exemplary embodiment;

FIGS. 3A and 3B include images of the current scan plane and the target scan plane with anatomy awareness in accordance with an exemplary embodiment;

FIGS. 4A and 4B illustrate images of the current scan plane and the target scan plane with context awareness in accordance with an exemplary embodiment;

FIG. 5 is a schematic of workflow of guided ultrasound scanning procedure in accordance with an exemplary embodiment; and

FIG. 6 is a flow chart illustrating a method for guided ultrasound imaging in accordance with an exemplary embodiment.

### DETAILED DESCRIPTION

As will be described in detail hereinafter, systems and methods for ultrasound imaging are presented. More particularly, the systems and methods are configured for guided ultrasound imaging suitable for low-cost ultrasound imaging equipment.

The terms “subject” and “patient” are used equivalently and interchangeably to refer to a person who is being examined by an ultrasound scanning procedure. The term “ultrasound probe” refers to a sensor of the ultrasound scanning device that is moved over an organ of interest of the subject to acquire ultrasound signals. The term “image” refers generally to the data acquired by the ultrasound probe and specifically to the two-dimensional image generated by a processor using the data obtained during the ultrasound scanning procedure. The term “plurality of images” refers to a series of images obtained. during the ultrasound scanning procedure. The term ‘target organ’ refers to an anatomical organ of interest. The term “pose” refers either to a position of the ultrasound probe that acquires an image or a position of a target organ within the image. The position of the probe or the target organ may be specified by one or more angles, co-ordinates of the probe in a three-dimensional space or co-ordinates of the portion of the anatomical image in a two dimensional space and a time of acquisition of the image. The term scan plane refers to a plane in a three dimensional space that provides data acquired during the scanning procedure. The term “standard scan plane” refers to any scan plane used as reference image for diagnostic purposes. The term “standard pose” refers either to a pose of the ultrasound probe or a desirable pose of the target organ within a standard scan plane. The term ‘scan plane’ is used to denote an image plane in the subsequent paragraphs. Specifically, the term ‘acquired scan plane’ is a scan plane corresponding to the acquired image. The terms “expected image” and “desired image” are used equivalently and interchangeably to refer to an acceptable image in the proximity of a standard scan plane. The term “anatomical context” in an image refers to a number of attributes of the images such as, but not limited to, a number of image segments in the image, a list of organs that are seen in the image segments of the image, relative positions of the image segments with respect to one or more other image segments, and relative properties of the image segments with reference to corresponding properties in a standard scan plane image. The term “image segmentation” refers to identifying a plurality of organs in an image and determining corresponding boundaries within the image plane.

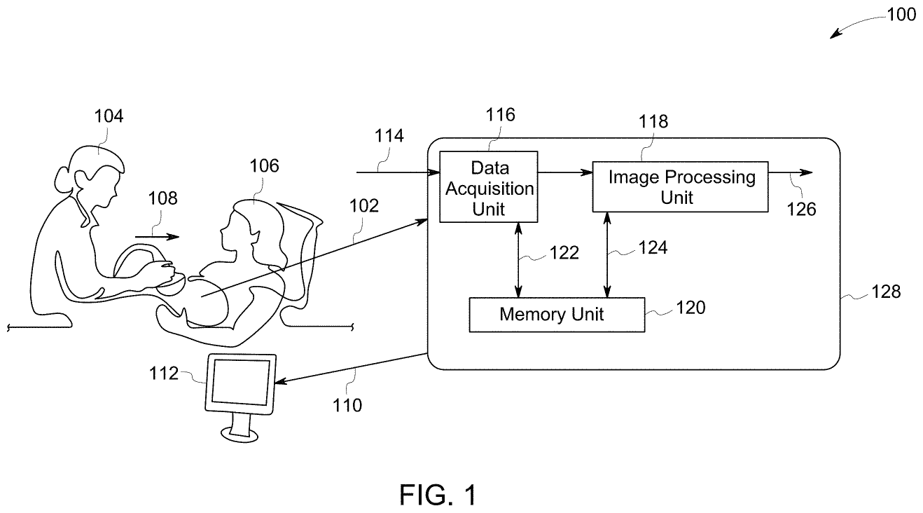

FIG. 1 is a diagrammatic illustration of an ultrasound scanning system 100 for guided ultrasound imaging in accordance with an exemplary embodiment. The ultrasound scanning system 100 includes an ultrasound probe 108 configured to acquire an image of a target organ of a subject 106 during an ultrasound scanning procedure. The acquired image corresponds to a pose of the target organ. The ultrasound probe 108 is configured to acquire a plurality of images 102 when operated by an operator (or a medical professional) 104 by moving over the target organ. The plurality of images 102 may be stored in a memory unit or displayed on a display device 112.

The ultrasound scanning system further includes a guidance unit 128 communicatively coupled to the ultrasound probe 108 and configured to receive the plurality of images 102. The guidance unit 128 is further configured to receive one or more scanning parameters 114 from an operator. In one embodiment, the guidance unit 128 is configured to process the plurality of images 102 received from the ultrasound probe 108 using the one or more scanning parameters 114 and generate guidance for the operator 104 to continue and complete the scanning procedure. In one embodiment, the ultrasound scanning may be directed to examine the abdominal area and in such an embodiment, one of the scanning parameters 114 may be indicative of one or more organs abdomen. As another more specific example, the ultrasound scanning may be required to be directed to examine kidneys. In such a case, one of the scanning parameters 114 may be indicative of kidneys as target organ. The guidance unit 128 is configured to process the received plurality of images 102 to verify if scanning is directed to the intended target organ. If the scanning is not directed to the intended target organ, a guidance 110 is generated by the guidance unit 114 and presented to the operator 104 through the display device 112.

In one embodiment, the guidance unit 128 includes a data acquisition unit 116, a memory unit 120 and an image processing unit 118 communicatively coupled to each other by communications bus lines 122, 124. In one embodiment, the data acquisition unit 116 includes electronic circuitry to establish communication with the ultrasound probe 108 and configured to receive the plurality of images 102. The data acquisition unit 116 may be further configured to perform pre-processing of the acquired data for noise reduction, transient removal and other such data conditioning operations. The pre-processed data from the data acquisition unit 116 may be stored in the memory unit 120 for further use by other modules of the image guidance unit 128. In one embodiment, the data acquisition unit 116 is also configured to acquire the scanning parameter 114 from a user through a key board or any other input device. The data acquisition unit 116 may also assist the user to feed correct values of the scanning parameter 114 by providing options displayed via the display device 112. In one embodiment, the data acquisition unit 116 is further configured to provide one of the plurality of acquired images to the image processing unit 118 for further processing or to the memory unit 120 for storage purposes.

The memory unit 120 is communicatively coupled to the data acquisition unit 116. The memory unit 120 may be a single memory storage unit or a plurality of smaller memory storage units coupled together to work in a coordinated manner. In one embodiment, the memory unit 120 may be a random-access memory (RAM), read only memory (ROM), or a flash memory. The memory unit 120 may also include, but not limited to, discs, tapes, or hardware drive based memory units. It may be noted that a part of the memory unit 120 may also be disposed at a remote location either as a hardware unit or as a cloud service providing computational and storage services. In one embodiment, the memory unit 120 may be pre-loaded with anatomical information such as an atlas corresponding to a plurality of organs that may he examined by ultrasound image techniques. The anatomical information may be labelled with a plurality of attributes such as, but not limited to, age, region, gender and medical conditions of subjects. The memory unit 120 may also be configured to store meta data related to the anatomical information that may be used to determine context awareness with in an acquired image. In one embodiment, the memory unit 120 may include a non-transitory computer readable medium having instructions to enable at least one processor module to provide scanning guidance in accordance with exemplary embodiments of the present specification.

The image processing unit 118 is communicatively coupled to the memory unit and configured to process one or more of the plurality of images 102 received from the data acquisition unit 116 or retrieved from the memory unit 120. The image processing unit 118 may include a graphical processing unit (GPU), one or more microprocessors, and a microcontroller. The image processing unit 118 further includes specialized circuitry or hardware such as, but not limited to, a field programmable gate array (FPGA), application specific integrated circuit (ASIC). In one embodiment, the image processing unit 118 is configured to select one of the recent images among the plurality of acquired images and perform image segmentation to generate a segmented image. The guidance unit 128 is also configured to identify at least one of an organ a dimension corresponding to the organ, or a location of the organ based on the segmented image.

The image processing unit 118 is further configured to determine an anatomical context 126 based on the acquired image. The acquired image corresponds to a scan plane in 3D volume of the patient anatomy. The scan plane corresponds to the pose of the target organ within the acquired image. The image processing unit 118 is also configured to determine a relative location of the scan plane with reference to a standard scan plane corresponding to a standard pose of the target organ. The standard scan plane referred herein is an anatomical scan plane such as, but not limited to, a transversal plane, a sagittal plane, a parasagittal plane or a coronal plane. Further, the image processing unit 118 may be configured to determine one or more organs adjacent to the target organ within the acquired image. The list of adjacent organs, their relative positions and sizes may be useful to determine an anatomical context of the target organ.

The image processing unit 118 is further configured to determine an orientation of the acquired image with reference to the plurality of standard scan planes. The image processing unit 118 is also configured to generate a scanning guidance based on the relative location of the scan plane. The scanning guidance includes information to move the probe towards the standard pose. The image processing unit 118 is further configured to determine a relative parameter corresponding to the organ based on one or more of the dimension of the organ and the location of the organ. The image processing unit 118 is also configured to determine a displacement value and a direction of displacement with reference to the standard scan plane. The image processing unit 118 is further configured to generate a scanning guidance based on the relative location of the scan plane. The scanning guidance includes information required to move the probe towards the standard pose. The image processing unit 118 may also be configured to relate the pose of the target organ in an image to a pose of the probe. In one embodiment, the guidance 110 may be in the form of movements to be made by the operator. For example, the scanning guidance may include a direction and an extent of desired movement for the ultrasound probe 108. In another embodiment, the guidance 110 may be in the form of deviation of the scanned images from the expected images. For example, the scanning guidance includes a measure of proximity of the acquired image from the desired image. In yet another embodiment, the guidance 110 may be in the form of an alert signal to precisely guide the scanning to acquire desired plurality of images 102. The alert signal may be in the form of an audio signal, a visual signal or a tactile signal.

The ultrasound scanning system 100 further includes an output device communicatively coupled to the guidance unit 128 and configured to present the scanning guidance 110 for aiding continuance of the scanning procedure. In one embodiment, the output device may be a display device configured to present the movements to be made by the operator. In another embodiment, the output device may be a loud speaker device presenting audio signals to the operator. In yet another embodiment, the output device may be a tactile device configured to present a tactile signal to the operator during the scanning procedure. In an exemplary embodiment, the display device 112 is configured to display the direction and the extent of desired movement for the ultrasound probe.

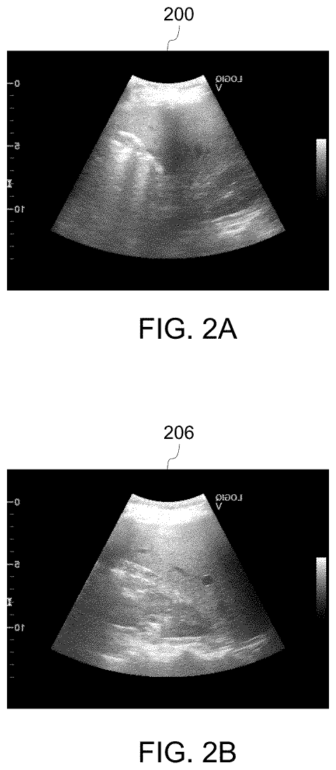

FIG. 2A illustrates an acquired image 200 in comparison with a standard scan plane image 206 of FIG. 2B in accordance with an exemplary embodiment. The acquired image 200 is processed by the image processing unit 118 (as shown in FIG. 1) to identify images of organs that may be found within the image. The image processing unit 118 is further configured to analyze the relative size and positions of organs within the image to determine a standard scan plane proximal to the acquired image 200. The anatomical awareness information corresponding to the standard scan plane is also retrieved or analyzed by the image processing unit 118. Further, the image processing unit 118 is configured to compare the relative size, shape and positions of organs within the acquired image with the standard scan plane image 206 to generate scanning guidance. In the illustrated embodiment, the acquired image 200 is generated during kidney scanning procedure from the ultrasound probe 108 (as shown in FIG. 1). The image processing unit 118 may initially identify images of kidney and possibly one or more of liver and bowel. In an embodiment where images of kidney and liver are identified, the scan plane corresponding to the acquired image is considered as closer to a traverse image. The image processing unit 118 is further configured to retrieve the transverse image having images of a liver at the top portion and an image of the kidney at the bottom portion as the standard scan plane image 206. Further, the image processing unit 118 is configured to compare the images of kidney and liver in the acquired image 200 with corresponding organs in the standard scan plane image 206 to determine guidance to the operator.

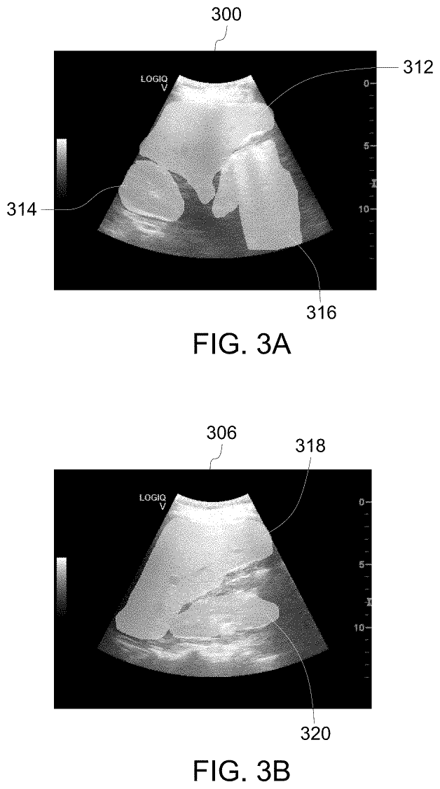

FIG. 3A includes a segmented image 300 and a corresponding standard scan plane image 306 of FIG. 3B with anatomy awareness in accordance with an exemplary embodiment. The segmented image 300 is a segmented version of the acquired image 200 of FIG. 2. In one embodiment, a conventional image segmentation technique may be employed by the image processing unit 118 (as shown in FIG. 1) to generate the segmented image 300. In another embodiment, a deep learning network may be used, by the image processing unit 118 to generate the segmented image 300. In the illustrated embodiment, the segmented image 300 includes a liver segment 312, a kidney segment 314 and a bowel segment 316 arranged in a triangular pattern. The liver segment 312 is larger in size compared to kidney segment 314 and bowel segment 316. The bowel segment 316 is prominently seen and the kidney segment 314 is smaller in size disposed on the left side of the segmented image 300. The FIG. 3B corresponding to a standard scan plane image 306 is a segmented transverse image. The standard scan plane image 306 includes a liver segment 318 and a kidney segment 320 disposed below the liver segment 318. It may be noted that the bowel segment 316 is completely absent in the standard scan plane image 306. The kidney segment 320 is shifted version of the kidney segment 314. The liver segment 318 is larger than the liver segment 312 in the image 300. The segmented image 300 and the corresponding standard scan plane image 306 provides a basis for generating the guidance in embodiments of the present specification as explained in subsequent figures.

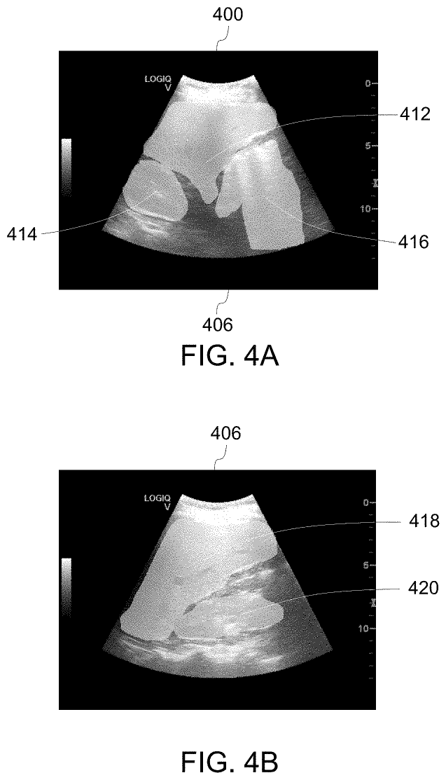

FIG. 4A illustrates the segmented image 400 and the corresponding standard scan plane image 406 of FIG. 4B with context awareness in accordance with an exemplary embodiment. The segmented image 400 corresponds to the segmented image 300 of FIG. 3A, Similarly, the standard scan plane image 406 corresponds to the segmented transverse image 306 of FIG. 3B. The segmented image 400 and the corresponding standard scan plane image 406 further includes additional information about size and location of liver and kidney segments. Specifically, the segmented image 400 includes reference locations 412, 414, 416 corresponding to the liver segment, kidney segment and bowel segment of the image 400. Similarly, the segmented image 406 includes reference locations 418, 420 corresponding to the liver segment and the kidney segment of the segmented image 406, The distance values between the reference locations 412, 414 and 416 are also illustrated in the image 400. Similarly, the distance values between the reference locations 418 and 420 is indicated in the image 406. In one embodiment, the co-ordinates of the reference locations 412, 414, 416, 418, 420 may be used to determine context of the acquired image 400 with reference to the standard scan plane image 406. In another embodiment, the relative distance values between the reference locations 412, 414, 416 and 418, 420 may also be used to determine the context information of the segmented image 400 with reference to the standard scan plane 406.

In one embodiment, the segmented image 400 and the segmented standard scan plane image 406 are determined by the image processing unit 118 using any of the known segmentation techniques. In some embodiments, the segmented standard scan plane image 406, anatomical information corresponding to the segmented standard scan plane image 406 may be available in the memory unit.

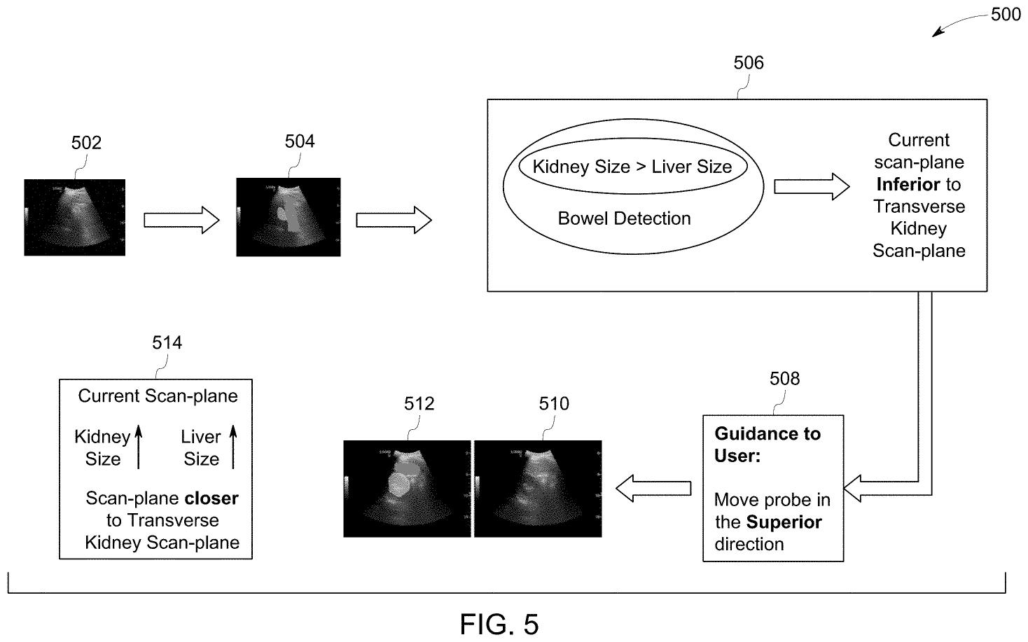

FIG. 5 is a schematic 500 of workflow of guided ultrasound scanning technique in accordance with an exemplary embodiment. The schematic 500 illustrates an input image 502 selected from a plurality of images obtained during an ultrasound scanning procedure based on a time-stamp value. The illustrated embodiment is related to a kidney scanning and aim of scanning is to acquire transverse kidney scan plane. The input image 502 is processed by the image processing unit 118 of FIG. 1 to generate anatomy awareness information. In one embodiment, the anatomy awareness information may be obtained from a segmented image 504 generated by the image processing unit 118. It may be noted herein that the image segmentation may be aided by additional information provided by the operator about the purpose of scanning procedure. Any one of the conventional image segmentation techniques such as model image segmentation, heuristics-based image segmentation, a deep learning based image segmentation technique may be used to generate the segmented image 504.

The segmented image 504 may be further processed using the image processing unit 118 based on one or more standard scan plane images to generate a context awareness information. In one embodiment, the context awareness information 506 may be in the form of relative positions, relative sizes and relative distance values between the adjacent image segments within the segmented image 504 with reference to the positions of segments in the standard scan plane image (not shown in the schematic 500). In the illustrated embodiment, the context awareness information includes relatively smaller kidney size compared to liver size and indicates that the current scan plane is inferior to transverse kidney scan plane. The schematic 500 also illustrates generation of scan guidance in block 508. The scan guidance generation includes, but not limited to, a direction of movement and a tilting angle for the ultrasound scanning probe. In the illustrated embodiment, the scan guidance is to move the probe in a superior direction. The scan guidance may be provided in the form of a visual signal, an audio signal or a tactile signal. The operator of the ultrasound scan probe receives the guidance and moves the probe towards a more desirable position for acquiring a new scanning image 510. Further, the newly acquired scanning image 510 may be processed using the same workflow 500 to continue the scanning procedure. It may be noted that subsequent images 514 acquired from the ultrasound probe using the work flow 500 are nearer to standard scan plane 512 images. During the progress of the scanning, the relative sizes of the segments may be analyzed to verify that sizes of image segments towards convergence. Further, the relative sizes of detected organs may also be verified to ensure that the acquired images are nearer to standard scan planes. In the illustrated embodiment, the kidney and the liver segments are monitored to ensure that their sizes are increasing during guided scanning. Additionally, the size of the bowel segment may also be verified to be decreasing in size as the operator is able to move the ultrasound probe to acquire the standard scan plane image.

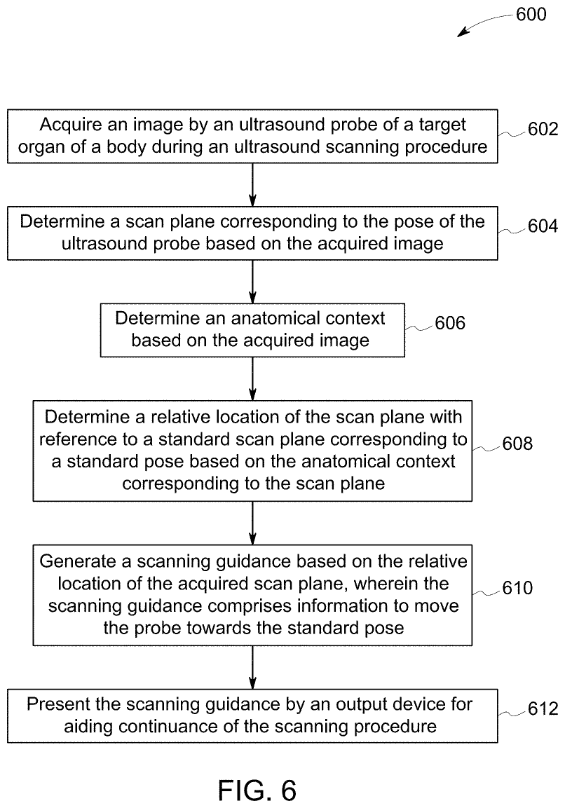

FIG. 6 is a flow chart 600 illustrating a method for guided ultrasound imaging in accordance with an exemplary embodiment. The flow chart 600 specifically illustrates scanning guidance method that includes acquiring an image by an ultrasound probe of a target organ of a body during an ultrasound scanning procedure in step 602. The acquired image corresponds to a pose of the target organ. Acquiring the image also includes acquiring a plurality of images by the ultrasound probe moving over the target organ and storing the plurality of images.

The method 600 further includes processing the image by a guidance unit to determine a scan plane corresponding to the pose of the target organ based on the acquired image at step 604. The processing of the image by the guidance unit further includes determining an anatomical context based on the acquired image at step 606. The processing of the image also includes determining a relative location of the scan plane with reference to a standard scan plane corresponding to a standard pose based on the anatomical context corresponding to the scan plane at step 608. The processing of the image also includes generating a scanning guidance at step 610 based on the relative location of the scan plane. The scanning guidance includes information to move the probe towards the standard pose.

The processing includes determining orientation of the acquired image with reference to a plurality of anatomical planes. The anatomical planes include a transverse plane, a sagittal plane, a parasagittal plane, and a coronal plane. Further, the processing includes identifying a standard scan plane corresponding to the scan plane and acquiring the standard scan plane from a memory unit. The processing includes performing image segmentation corresponding to the acquired image to generate a segmented image. The processing includes identifying at least one of an organ in the acquired image, a dimension corresponding to the organ and a location of the organ in the acquired image based on the segmented image. The processing further includes determining a relative parameter corresponding to the organ based on one or more of the dimension of the organ and the location of the organ. Determining the relative location of the scan plane includes determining a displacement value and a direction of displacement with reference to the standard scan plane. Generating the scanning guidance includes determining a direction and an extent of desired movement for the ultrasound probe.

The method of ultrasound scanning also includes presenting the scanning guidance on an output device at step 612 for aiding continuance of the scanning procedure. Presenting the scanning guidance comprises displaying the direction and the extent of desired movement of the probe on a display device.

Embodiments of the present specification obviate the use of any external sensor such as camera, accelerometer, gyroscope, electromagnetic sensor to guide the user obtain the clinically correct scan plane. Embodiments presented herein enable relatively less experienced or new users to deploy ultrasound with ease. Specifically, the technique reduces the skill barrier by assisting the user regardless of their level of experience to quickly acquire the clinically correct images. Thus, the techniques presented herein help in increasing available market and according to one estimate, a tenfold increase in market access is estimated in African markets.

It is to be understood that not necessarily all such objects or advantages described above may be achieved in accordance with any particular embodiment. Thus, for example, those skilled in the art will recognize that the systems and techniques described herein may be embodied or carried out in a manner that achieves or improves one advantage or group of advantages as taught herein without necessarily achieving other objects or advantages as may be taught or suggested herein.

While the technology has been described in detail in connection with only a limited number of embodiments, it should be readily understood that the specification is not limited to such disclosed embodiments. Rather, the technology can be modified to incorporate any number of variations, alterations, substitutions or equivalent arrangements not heretofore described, but which are commensurate with the spirit and scope of the claims. Additionally, while various embodiments of the technology have been described, it is to be understood that aspects of the specification may include only some of the described embodiments. Accordingly, the specification is not to be seen as limited by the foregoing description but is only limited by the scope of the appended claims.

1. An ultrasound scanning guidance method comprising:
acquiring an image by an ultrasound probe of a target organ during an ultrasound scanning procedure, wherein the acquired image corresponds to a pose of the target organ in an acquired scan plane;
processing the image by a guidance unit to:
determine an anatomical context around the target organ based on the acquired image;
determine a relative location of the acquired scan plane with reference to a standard scan plane based on the pose of the target organ and the anatomical context;
generate scanning guidance based on the relative location of the acquired scan plane, wherein the scanning guidance comprises information to move the probe towards a standard pose of the target organ; and
presenting the scanning guidance by an output device for aiding continuance of the scanning procedure.

2. The method of claim 1, wherein the processing comprises determining an orientation of the target organ seen in the acquired image with reference to a plurality of anatomical planes comprising a transverse plane, a sagittal plane, a parasagittal plane, and a coronal plane.

3. The method of claim 1, wherein acquiring the image comprises acquiring a plurality of images by the ultrasound probe moving over the target organ and storing the plurality of images.

4. The method of claim 1, wherein the processing comprises identifying a standard scan plane corresponding to the acquired scan plane and acquiring the standard scan plane from a memory unit.

5. The method of claim 1, wherein the processing comprises performing image segmentation corresponding to the acquired image to generate a segmented image.

6. The method of claim 5, wherein the processing comprises identifying at least one of an organ in the acquired image, a dimension corresponding to the organ, or a location of the organ in the acquired image based on the segmented image.

7. The method of claim 5, further comprising determining a relative parameter corresponding to the organ based on one or more of a dimension of the organ and a location of the organ.

8. The method of claim 1, wherein determining the relative location of the acquired scan plane comprises determining a displacement value and a direction of displacement with reference to the standard scan plane.

9. The method of claim 1, wherein generating the scanning guidance comprises determining a direction and an extent of desired movement for the ultrasound probe.

10. The method of claim 9, wherein presenting the scanning guidance comprises displaying the direction and the extent of desired movement on a display device.

11. An ultrasound scanning system, comprising:
an ultrasound probe configured to acquire an image of a target organ during an ultrasound scanning procedure, wherein the acquired image corresponds to a pose of the target organ in an acquired scan plane;
a guidance unit communicatively coupled to the ultrasound probe and configured to:
determine an anatomical context around the target organ based on the acquired image;
determine a relative location of the acquired scan plane with reference to a standard scan plane based on the pose of the target organ and the anatomical context;
generate a scanning guidance based on the relative location of the acquired scan plane, wherein the scanning guidance comprises information to move the probe towards a standard pose of the target organ; and
an output device communicatively coupled to the guidance unit and configured to present the scanning guidance for aiding continuance of the scanning procedure.

12. The system of claim 11, wherein the guidance unit is further configured to determine an orientation of the target organ in the acquired image with reference to a plurality of anatomical planes comprising transverse, sagittal, parasagittal, and coronal planes.

13. The system of claim 11 wherein the ultrasound probe is configured to acquire a plurality of images by the ultrasound probe moving over the target organ and storing the plurality of images.

14. The system of claim 11, wherein the guidance unit is configured to identify a standard scan plane corresponding to the acquired scan plane and acquiring the standard scan plane from a memory unit.

15. The system of claim 11, wherein the guidance unit is configured to perform image segmentation corresponding to the acquired image to generate a segmented image.

16. The system of claim 15, wherein the guidance unit is configured to identify at least one of an organ in the acquired image, a dimension corresponding to the organ, or a location of the organ in the acquired image based on the segmented image.

17. The system of claim 15, further configured to determine a relative parameter corresponding to the organ based on one or more of a dimension of the organ and a location of the organ.

18. The system of claim 11, wherein the guidance unit is configured to determine a displacement value and a direction of displacement with reference to the standard scan plane.

19. The system of claim 11, wherein the guidance unit is configured to determine a direction and an extent of desired movement for the ultrasound probe.

20. A non-transitory computer readable medium having instructions to enable at least one processor module to:
acquire an image by an ultrasound probe of a target organ during an ultrasound scanning procedure, wherein the acquired image corresponds to a pose of the target organ in an acquired scan plane;
process the image by a guidance unit to:
determine an anatomical context around the target organ based on the acquired image;
determine a relative location of the acquired scan plane with reference to a standard scan plane corresponding to a standard pose of the target organ based on the anatomical context and the pose the target organ seen in the acquired scan plane;
generate a scanning guidance based on the relative location of the acquired scan plane, wherein the scanning guidance comprises information to move the probe towards the standard pose of the target organ; and
present the scanning guidance on an output device for aiding continuance of the scanning procedure.
